(19) United States
(12) Patent Application Publication
(10) Pub. No.: US 2021/0252454 A1
(43) Pub. Date: Aug. 19, 2021

(54) Optimized Biomass Combustion (Burning) for Renewing Ecosystem, Environment, and Atmosphere

(71) Applicants: Venkata Ramana Chaganti, (US); Murali Krishna Cheruvu, (US)

(72) Inventors: Venkata Ramana Chaganti, McDonough, GA (US); Murali Krishna Cheruvu, Irving, TX (US)

(21) Appl. No.: 17/224,919

(22) Filed: Apr. 30, 2021

(51) Int. Cl. B01D 53/76; A61K 35/38; A61K 35/20; C02F 1/28; B01D 53/56; B01D 53/62; B01D 53/72; B01D 53/50

(52) U.S. Cl. CPC B01D 53/76; A61K 35/38; A61K 35/20; C02F 1/283; C02F 2103/007; B01D 53/62; B01D 53/72; B01D 53/50; B01D 53/56

(57) ABSTRACT

This invention “Optimized Biomass Combustion (Burning) for Renewing Ecosystem, Environment, and Atmosphere” is a process in which pure cow ghee is burned using sticks of specific trees along with other biomass such as dried cow dung cakes, grains, metal dust, herbs, etc., in a specially designed open brazier (combustion chamber). The amount of ghee, biomass, dimensions of the combustor, and length of time depends on the purposes that are given below. Reduce indoor air pollution and outdoor (ambient) air pollution caused by vehicles, and industries. Reduce rainwater and waterbodies pollution, curtail agriculture soil pollution. Mitigate intensities of hurricanes, storms, and tornadoes. Recovery from droughts, suppression of forest wildfires, mitigate virus/bacteria count, increase immunity, and reduce mosquitoes (and mosquito eggs) in the proximity.

### BACKGROUND OF THE INVENTION

Air Pollution, Global Warming, Water Pollution, Wildfires, Droughts, Viruses/Bacteria spread are major environmental and atmospheric challenges that the ecosystem/world is facing today.
1. In United States of America about 655,000 people die every year due to heart attack and costs about $200 billion. From 1980 to 2014, the mortality rate increased 29.7 percent due to respiratory related diseases in United States of America.
2. Reports indicate that in 2012 there were about 40.6 million United States of America adults who used herbs and supplements as Complementary and Alternative Medicine (CAM).
3. Pollution is everywhere and everybody's concern as recent studies indicate that on average about 8.7 million people die every year due to air pollution.
4. Nitrogen Oxides (NOₓ) are one of the major air pollutants and the top two contributors of NOₓ pollution are vehicular/road transportation and Energy production and distribution.
5. About 50,000 people of United States of America visit hospitals due to the consumption of accidental Carbon Monoxide (CO) poisoning.
6. Usage of Volatile Organic Compounds (VOCs) were reduced to 50 percent between 1970 and 2020, still about 15 million tons were produced in 2020 and the usage was considered high. Major sources of VOC pollution are wildfires and vehicles.
7. Sulfur Dioxide (SO₂) is directly associated with various adverse health effects. While SO₂ usage is reduced by 80 percent from 23.1 million tons in 1990 to 4.5 million tons in 2014, the majority of SO₂ emissions are coming from fuel combustion that remain as a major concern.
8. Carbon Dioxide (CO₂) emission is very critical given that CO₂ represents about 81 percent of the greenhouse gases. Greenhouse Gas Index, a measure to global warming, was increased to 1.37, which was about 37 percent increase from 1990 to 2015. About 80 percent of the carbon emissions are caused by developed countries such as United States of America indicating the urgency to find the solutions to net-zero emissions.
9. According to World Health Organization (WHO), about one percent of chest infections, five percent of lung cancers, and two percent of lung and heart diseases are due to ambient (outdoor) pollution.
10. Majority of the studies agree that industrial pollution is a major culprit of the global warming and impacting health of over 100 million people in the world.
11. Every aspect of the Energy such as electricity production and distribution organizations contribute to ambient (outdoor), water, and land pollution.
12. United States of America vehicles and road transportation constitute over 55 percent Nitrogen Oxides (NOₓ) emissions, about 10 percent Volatile Organic Compounds (VOCs) and less than 10 percent of particulate matters—PM_{2.5} and PM₁₀.
13. According to United States of America Environmental Agency some of the air pollutants are two to five times more in indoors than the outdoor concentrations and a typical American spends about 90 percent of the time in indoors.
14. Three key aspects—floods, droughts, and storms contribute to 80 to 90 percent of the world disasters. Droughts effect yearly about 55 million people in the world.
15. Acid rains are caused by Sulfur Dioxide (SO₂) and Nitrogen Oxides (NOₓ) with a pH value between 4.2 and 4.4.
16. In the world there are about 750 million people who do not have clean water to drink and in every two minutes a kid dies due to diarrhea.

Page 4 of 26
17. About 13 million United States of America households get the drinking water from the groundwater such as wells. Pollution from fertilizers and pesticides can contaminate groundwater.
18. In United States of America, 2020 ended up as seventh expensive hurricane season with $47 billion from 25 storms and 6 category 3 or stronger hurricanes costing 430 human lives.
19. About 1,000 tornadoes hit every year in Tornado Alley of United States of America and about 20 of them expected to be EF-3 intensity with estimated winds over 200 mph. In 2020 alone, there were 1,075 tornadoes costing 76 human lives and $36 million.
20. In 2020 alone, there were 57,000 wildfires in United States of America burned more than 10.3 million acres.
21. About 400,000 people were killed in United States of America due to COVID-19 pandemic virus.
22. Mosquitoes are seasonal and cause illness such as malaria in humans. Every two minutes, due to malaria, one child dies as per United Nations International Children's Emergency Fund (UNICEF) report.
23. Studies have observed in recent years that the overall immunity system in human beings is getting weakened, particularly people at ages between 50 and 59.

### BRIEF SUMMARY OF THE INVENTION

This invention is used for the following purposes:
1. Reduce ambient (outdoor) air pollution
2. Reduce indoor air pollution in residential and commercial buildings
3. Reduce air pollution caused by vehicles
4. Reduce air pollution caused by industries
5. Reduce water pollution by decreasing Biological Oxygen Demand (BOD) levels and improving water quality aspects such as Total Dissolved Solids (TDS), pH, hardness, etc.
6. Curtail agriculture soil pollution by improving the soil quality
7. Mitigate the intensities of hurricanes, storms, and tornadoes
8. Recovery from droughts
9. Suppression of forest wildfires by creating favorable circumstances for clean (non-acidic) rainfall
10. Reduce virus/bacteria count
11. Increase immunity in humans, animals, and plants
12. Reduce the spread of mosquitoes (and the eggs of mosquitoes)

Combustion (burning) of wood sticks/pellets/chips from trees such as mango, pine, etc., and/or dried cow dung cakes with pure cow ghee in an open combustion chamber (brazier) generate vapors/smokes that help in achieving the above listed benefits. To improve the effects and for different purposes as mentioned above, various organic materials such as herbs, dry fruits, saffron, honey, butter, cow milk, goat milk, sandal wood, lentils, grains, etc., and metals such as gold, silver, etc., are burnt along with the wood sticks and pure cow ghee. The vapors/smokes in the form of particles (nano/atomic/molecules) produced in this process spread in the proximity with great kinetic energies and cause the desired results listed above.

Each of the above listed purpose is different from the other purpose and is achieved by varying the metrics such as:
1. The duration of the interval between the burning periods
2. Amount of biomass burned in each interval
3. Total time and amount of biomass burned
4. Dimensions of the combustion chamber (brazier)

### BRIEF DESCRIPTION OF SEVERAL VIEWS OF THE DRAWING

FIG. 1A shows the lateral view of the Brazier (Combustion Chamber). This specially designed Combustion Chamber can be made of a metal such as iron, copper, silver, or gold. It can also be build using earthen materials such as clay or bricks. The shape of the Combustion Chamber would be an open top inverted truncated hollow pyramid with lateral and closed bottom surfaces. The top and bottom surfaces are square shaped with the lateral surface being a trapezium. The height of the Combustion Chamber is equal to the length of the side of the top. The length of the side of the bottom is one-fourth (¼) the height of the Combustion Chamber or one-fourth (¼) the side of the open top. The dimensions of the Combustion Chamber depend on the purpose (listed in description) and the proximity to cover. Different shapes of Combustion Chamber such as triangular, circular, semi-circular, etc., can be used with the same ratios in the sizes as defined in FIG. 1A. However, inverted truncated pyramid with square top and bottom would be efficient for this process.

FIG. 1B shows the top view of the Brazier (Combustion Chamber that is shown in FIG. 1A).

FIG. 2 shows the open room (or shelter) to keep the Brazier (Combustion Chamber that is shown in FIG. 1A). This room must meet all fire resistance regulations given by the local authorities. This room is like a cabana in shape and having enough space to accommodate the Combustion Chamber, other materials, and should provide protection from wind, rain, etc. The sides of the room must be kept open but closed with grilled windows for the combusted vapors/smokes to spread easily into the atmosphere and keep away from animals, birds, and unauthorized people to enter. Ideally the Combustion Chamber is placed in the middle of the room.

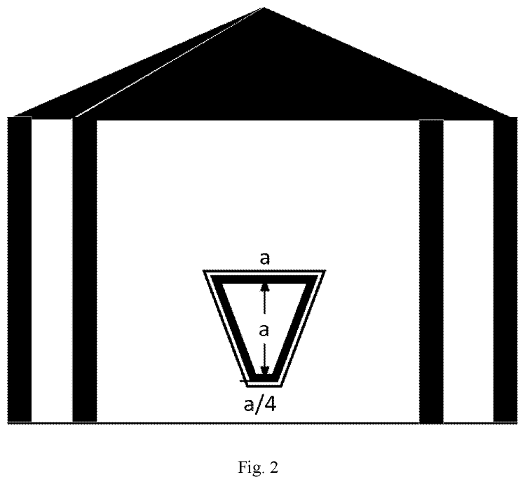

### DETAILED DESCRIPTION OF THE INVENTION

This invention is used for the following purposes.
1. Reduce ambient (outdoor) air pollution
2. Reduce indoor air pollution in residential and commercial buildings
3. Reduce air pollution caused by vehicles
4. Reduce air pollution caused by industries
5. Reduce water pollution by decreasing Biological Oxygen Demand (BOD) levels and improving water quality aspects such as Total Dissolved Solids (TDS), pH, hardness, etc.
6. Curtail agriculture soil pollution by improving the soil quality
7. Mitigate the intensities of hurricanes, storms, and tornadoes
8. Recovery from droughts
9. Suppression of forest wildfires by creating favorable circumstances for clean (non-acidic) rainfall
10. Reduce virus/bacteria count
11. Increase immunity in humans, animals, and plants
12. Reduce the spread of mosquitoes (and the eggs of mosquitoes)

Combustion (burning) of wood sticks/pellets/chips from trees such as mango, pine, etc., and/or dried cow dung cakes with pure cow ghee in an open combustion chamber (brazier) generate vapors/smokes that help in achieving the above listed benefits. To improve the effects and for different purposes as mentioned above, various organic materials such as herbs, dry fruits, saffron, honey, butter, cow milk, goat milk, sandal wood, lentils, grains, etc., and metals such as gold, silver, etc., are burnt along with the wood sticks and pure cow ghee. The vapors/smokes in the form of particles (nano/atomic/molecules) produced in this process spread in the proximity with great kinetic energies and cause the desired results listed above.

Each of the above listed purpose is different from the other purpose and is achieved by varying the metrics such as:
1. The duration of the interval between the burning periods
2. Amount of biomass burned in each interval
3. Total time and amount of biomass burned
4. Dimensions of the combustion chamber (brazier)

This invention has two processes that may be used together or independently—(a) Biomass Combustion (Burning) Process and (b) Thermodynamic Fireball Process.

### A. Biomass Combustion (Burning) Process

Combustion (burning) of wood sticks (or wood chips or wood pallets), from trees such as mango, pine, etc., and/or dried cow dung cakes with pure cow ghee in a combustion chamber (or brazier) for certain periods of time in certain intervals produce vapors/smokes in the form of particles (nano/atomic/molecules) spread in the proximity with great kinetic energies and fulfill the purpose listed above.

These vapor/smoke nano particles will reduce the pollution by interacting with the pollutants, greenhouse gases, reducing the germ (virus/bacteria) count, and reduce mosquitoes in and around the proximity. These vapor particles collect water vapor from the atmosphere and constitute favorable circumstances for a good rainfall. These particles, potentially, reduce the intensities of natural calamities—hurricanes, tornadoes, wildfires, droughts, and storms. The ashes after completion of the biomass combustion process can be used to treat waterbodies and improve the soil quality.

To improve the effects and for different purposes as listed above, various organic materials such as herbs, dry fruits, saffron, honey, butter, cow milk, goat milk, sandal wood, lentils, grains, etc., and metals such as gold, silver, etc., get burnt along with the wood sticks and pure cow ghee. The metrics such as—(a) how long (duration) the process benefits would last and (b) how far (distance) the process benefits can reach—would depend on various factors including the following: quantities and types of materials used, time intervals (and duration) of the process, and dimensions of the combustion chamber.

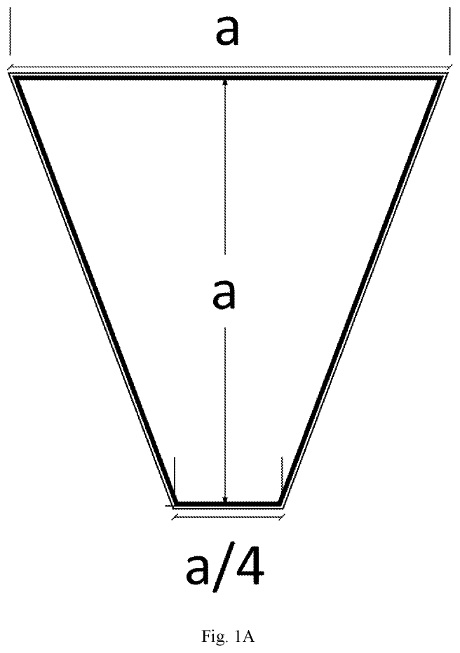

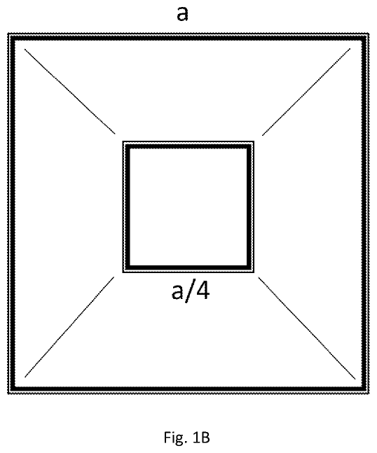

Biomass Combustion (Burning) Process Materials
1. Use specially designed Brazier (combustion chamber), as shown in FIG. 1, made of a metal like iron, copper, silver, or gold. It can also be build using earthen materials such as clay or bricks.
2. Ideal shape of the Brazier would be an open top inverted truncated hollow pyramid with lateral surfaces and closed bottom surface. The top and bottom surfaces are square shaped with the lateral surface being a trapezium. The height of the Brazier is equal to the length of the side of the top. The length of the side of the bottom is one-fourth (¼) the height of the Brazier or one-fourth (¼) the side of the open top.
3. The dimensions of the Brazier depend on the necessity such as the area/volume of the proximity (location) that requires the benefits (such as reducing ambient pollution).
4. The shape and dimensions of the room (or shelter) to keep the combustion chamber, as shown in FIG. 3, must meet all fire resistance regulations given by local authorities. The room/shelter is like a cabana in shape and having enough space to accommodate the brazier, other materials, and should provide protection from wind, rain, etc. The sides of the room must be kept open but closed with grilled windows for the combusted vapors/smokes to spread easily into the atmosphere and keep away from animals/birds/unauthorized people to enter. Ideally the combustion chamber is placed in the middle of the room.
5. Wood sticks (or wood chips or wood pallets) of appropriate sizes that fit well in the desired Brazier must be obtained, sized, and shaped from the specific trees. These wood sticks must not be invaded by the termites, must not be chemically treated, must be in enough dry condition.
6. Wood sticks would be collected from one or more of the following trees:
   a. Pine
   b. Mango
   c. Fig or Ficus Religiosa or Ashwath
   d. Cluster Fig or Ficus Glomerata or Udumbara
   e. Butea monasperma or Flame of the Forest or Palash
   f. Prosopis cineraria or Ghaf or Shami
   g. Capparis Spinosa or Vikadgand
7. Dried cow dung cakes of appropriate size can be used along with or without wood sticks.
8. Use pure organic cow ghee in liquid form. Organic cow ghee can be prepared from unpasteurized milk of the grass-fed cow. Boil the cow milk and when milk gets to warm temperature, ferment it to obtain the curd (yogurt). Buttermilk is prepared by churning the curd. Butter is obtained from the buttermilk and upon heating the butter we get the cow ghee. Reheat the cow ghee to get the liquid form—right before using it in the combustion (burn).
9. Camphor is kindled and is used to initiate the fire in the wood sticks. Use a few dry coconut pieces along with camphor.
10. Some processes may require one or more types medicinal herbs.
11. Some processes may require one or more types of dry fruits and/or nuts.
12. Some processes may require lentils and grains. Typical grains include wheat, paddy/rice, red lentil gram, green gram, yellow split peas or chana dal, soya gram, sesame, black Gram, and horse gram.
13. Some processes may require small quantities of metals such as gold, silver, and copper to be included in the form of metal dust.
14. Some processes may require raw unpasteurized honey.
15. Some processes may require fragrant materials such as sandal wood, saffron, and kasturi.
16. Some processes may require cow butter, cow milk, and goat milk.
17. Some processes may require sweet food materials like jaggery (made from sugar can juice), dates, honey, etc.

Biomass Combustion (Burning) Process Details
1. The biomass and organic materials—(a) pure cow ghee, (b) wood sticks and/or dried cow dung cakes (combinedly or separately referred as fuel), and (c) herbs, metals, dry fruits, sandal wood, lentils, grains, etc. (referred as material), as listed in the materials section—should be combined in appropriate proportion depending on the purpose. The details of the appropriate proportions of the materials remain as a trade secret.
2. Appropriate quantity of fuel (wood sticks and/or dried cow dung cakes) is placed in the brazier (combustion chamber).
3. Dry coconut hemi-spere holding camphor is placed in the brazier (combustion chamber)
4. Camphor pieces are kindled that initiates the combustion (burning).
5. Appropriate quantity (one unit) of liquid cow ghee is poured in the brazier (combustion chamber) on the fuel. For example, one unit could be 10 grams or 20 grams or prescribed amount of cow ghee that depends on the purpose chosen.
6. One unit of cow ghee is poured on the fuel at regular intervals of time (sprints). For example, one sprint could be 10 seconds or 20 seconds or prescribed interval of time that depends on the purpose chosen.
7. The amount of fuel, units of ghee, units of other materials, and number of sprints depend upon the purpose chosen.
8. Fuel is added to the brazier at regular intervals of time. The timing (when to dispense) and the amount of fuel/cow ghee/materials vary based on the following metrics:
   a. Dimensions of the brazier (Combustion Chamber)
   b. Type of wood sticks used
   c. Ambient Temperature
   d. The purpose chosen (such as reducing ambient pollution, etc.)
9. Fire is maintained in the brazier by pouring certain units of cow ghee, certain units of material, and certain quantity of fuel mentioned in (1).
10. The total time for combustion (referred as session) depends on purpose.
11. There can be a number of sessions in a day (Sunrise to Sunset).
12. The materials are combusted during daytime between Sunrise and Sunset as photochemical process during the daytime produces the required effects efficiently.
13. The process can be automated or done manually.
14. After the process is completed, the brazier is left for the materials to completely combust (burn) and turn into ashes, and vapors/smoke.
15. The ashes are let cool to room temperature and then collected in containers to be saved for the purpose to spread in appropriate waterbodies, and agricultural land.
16. The brazier (combustion chamber) is then cleaned and kept ready for the next session.
17. Please note that the combustion chamber and the shelter must meet all fire resistance regulations given by local authorities as the temperatures during the combustion process could reach in thousands of kelvin.

Biomass Combustion (Burning) Process Benefits
1. During this process, materials are burned to generate vapors (or fumes or smokes) in the form of nano/atomic/molecular particles that spread into the atmosphere with high kinetic energies.
2. Although these biomass combusted particles appear to increase the particulate matter for a short period, these particles are not only harmless but also help in reducing the existing pollution.
3. These kinetic nano/atomic/molecular particles can reduce the air pollution by interacting and combating with the pollutants, greenhouse gases, reduce the germs (viruses/bacteria) count and reduce the spread of mosquitoes (and eggs of mosquitoes).
4. These kinetic nano/atomic/molecular particles collect water vapors from the atmosphere and constitute favorable circumstances for clean and abundant rainfall.
5. Due to reduction of the pollutants in the atmosphere the rain thus obtained contains less Total Dissolved Solids (TDS).
6. Due to the interaction of these nano/atomic/molecular particles with Carbon Dioxide (CO₂) in the atmosphere, the acidity of the rainwater decreases and making the rainwater as non-acidic (pH close to 7) as possible.
7. These kinetic nano/atomic/molecular particles combat with natural calamities to reduce the intensities of hurricanes, storms, tornadoes, wildfires, and droughts.
8. The ashes are collected after completion of the biomass combustion (burning). These ashes are then dissolved in the waterbodies in appropriate quantities. These ashes contain many minerals that reduce the Biological Oxygen Demand (BOD) levels hence improve water quality.
9. Due to many minerals present in these ashes, soil quality gets improved when these ashes are spread (mixed) in agricultural lands.
10. Overall process increases the immunity in the living beings—humans, animals, and plants.
11. Organic cow ghee is a good source of energy; contains plenty of Omega-3 fatty acids and greatly works as antioxidant agent when consumed directly. However, when cow ghee is combusted the generated vapors/smokes are inhaled by the living beings in the proximity. These inhaled vapors/smokes directly enter bloodstreams and provide wider benefits along with increased immunity and resolves various health related problems such as heart attack, lung diseases, digestive system, brain functionality, etc.
12. When other organic biomass materials as explained in the process are combusted (burned) along with cow ghee resolve various biological, environmental, and atmospheric challenges including immunity vulnerabilities, pollution, and natural calamities—droughts, storms, hurricanes, tornadoes, and wildfires.
13. When herbs are combusted along with ghee, the vapors produced healing properties that are very useful for all living beings and the environment.
14. When metal dust such as silver, gold, etc., used in the combustion process along with cow ghee and other organic biomass materials, produce nano/atomic/molecular particles that are capable of cloud formation, rainfall, and destruction of pollutants such as Sulphur Dioxide (SO₂), etc.

### B. Thermodynamic Fireball Process

This is a thermodynamic process that generate very high Sun surface like temperatures in the range of 9,000 K (for few milli-seconds or so) by producing a fireball (fire column) for shorter periods (potentially up to one second) that can produce ultraviolet (UV) rays to mitigate viruses/bacteria. The fireball helps converting fatty acids in the cow ghee, high nutrient organic compounds present in cow milk and goat milk into vapors/smokes having kinetic energies capable of increasing immunity, reducing the bacteria/virus counts, reducing air, water, and soil pollution in and around the proximity. Fireball process can be conducted as a standalone process or can be augmented with the biomass combustion process for improved results.

Thermodynamic Fireball Process Materials and Details
1. The materials—(a) fresh cow milk, (b) fresh goat milk, (c) clay pot, (d) cow ghee, (e) fuel (wood sticks and/or dried cow dung cakes as mentioned in the Biomass Combustion Process), and (f) Brazier (Combustion Chamber).
2. Open empty clay pot is first heated to 100 degrees Celsius and then cow ghee is poured into the pot to fill it.
3. Then the heating is continued with the mouth of the clay pot kept opened till the cow ghee reaches the boiling point temperature at about 250 degrees Celsius.
4. At this juncture, appropriate quantities of fresh cow milk and fresh goat milk are collected in a long and wide spoon. This mixture of milks is poured carefully into the boiling ghee.
5. This act spontaneously generates a big fire ball (fire column) that can rise to a height of about 12 to 15 feet or more.
6. When cow milk and goat milk combination is poured in the boiling ghee, the water part (about 87 percent) present in the milk vaporizes instantly due to superheating. The change from liquid to vapor expands water to several hundred times and pushes the fire, ghee above it upwards that produces the fireball due to burning of ghee, and milk.
7. The amount of fuel, units of ghee, units of milk, clay pot dimensions, and dimensions of combustion chamber depend upon the purpose chosen.
8. Please note that the combustion chamber and the shelter must meet all fire resistance regulations given by local authorities as the temperatures during the combustion process could reach in thousands of kelvin.
9. This process should be done by mechanical (automation) or manually by experts in this field.

Thermodynamic Fireball Process Benefits

Along with the benefits listed in the Biomass Combustion Process, Thermodynamic Fireball Process produces beneficial sufficient low intensity ultraviolet (UV) rays that are capable of rapidly reducing the virus/bacteria count. This process produces vapors/smokes that are capable of increasing immunity in the human beings, reducing organic particulate pollution, air pollution and water pollution in and around the proximity.

### Unique Features

1. Shape of the Brazier (combustion chamber) would be an open top inverted truncated hollow pyramid with lateral surfaces and closed bottom surface. The top and bottom surfaces are square shaped with the lateral surface being a trapezium. The height of the Brazier is equal to the length of the side of the top. The length of the side of the bottom is one-fourth (¼) the height of the Brazier or one-fourth (¼) the side of the open top. We can use others shapes for top and bottom such as circular, semi-circular, triangular, etc., but meet the dimension criteria as mentioned above.
2. The dimensions of the Brazier depend on the requirements such as area/volume of the proximity (proximity) that maximizes the benefits of the scenarios such as reducing the ambient pollution spanning certain distance.
3. The overall process of burning (combusting) cow ghee with wood sticks along with or without the other materials (dried cow dung cakes, herbs, dry fruits, grains, etc.) is to generate vapors (or fumes or smokes) in the form of kinetic nano particles.
4. The length and the interval of the process vary in each of the scenarios explained in (2).

### Best Manufacturing Methods

1. Biomass Combustion Device can be built to automate the process
2. Biomass Combustion Device dimensions vary based on:
   a. Large structures to cover the scenarios such as industrial pollution
   b. Large structures to cover the scenarios such as hurricanes, storms, and tornadoes
   c. Large structures to cover large areas part of the green or smart city initiations
   d. Medium structures to address community parks, outside of the malls or public transportation places
   e. Medium to smaller structures to replace traditional fireplaces (at residential or commercial buildings)
   f. Smaller structures are ideal for individuals (personal scenarios)
3. Biomass Combustion Chamber and its paraphernalia can be installed at a fixed location or can be a movable one.
4. Pollution reducing Biomass Combustion Chamber can be installed at the following locations:
   a. At industries.
   b. In the community parks.
   c. In the city centers (like water tanks at city centers).
   d. In the residential or commercial buildings where the pollution reversal (reduction) vapors (or fumes or smoke) get spread by potentially using traditional fireplace.
   e. Possible reuse of existing AC (Air Conditioning) infrastructure in the buildings to spread the pollution controlling vapors (or fumes or smokes) generated by biomass combustion devices.
5. Purpose of the mobile biomass combustion device:
   a. Device can be installed into a vehicle with combustion chamber being on the top of the vehicle to spread the pollution reversal (reduction) fumes (or vapors or smokes) in the surroundings. These vehicles get driven in the heavy traffic locations.
   b. Devices can be installed into drones to make them go to the places such as middle of the waterbodies (such as rivers), huge agriculture fields, and into deep forests where mobility and reachability would otherwise be a challenge.
6. Biomass Combustion Devices that address natural calamities—hurricanes, storms, and tornadoes can be installed at the following locations:
   a. Prone to hurricane, storms, and tornadoes.
   b. Some sample locations in United States of America include in and around Tornado Valleys, Florida Coastline, Gulf of Mexico, Eastern Coast, and Western Coast.

1. Performing Biomass Combustion (Burning) or Thermodynamics Fireball Process on a daily basis with a prescribed metrics of substances (cow ghee, biomass, fuel, etc.) increases immunity, decreases major chronic and acute health problems in humans, and reduces viruses/bacteria/mosquitoes within the proximity.

2. Performing Biomass Combustion (Burning) or Thermodynamics Fireball Process on a daily basis with a prescribed metrics of substances (cow ghee, biomass, fuel, etc.) reduces indoor and ambient (outdoor) air pollution such as particulate matters (PM_{2.5}, PM₁₀), Nitrogen Oxides (NOₓ), Carbon Oxide (CO), Volatile Organic Compounds (VOCs), Sulphur Dioxide (SO₂), greenhouse gases—Carbon Dioxide (CO₂), Methane, and ground level Ozone (O₃) within the proximity.

3. Performing Biomass Combustion (Burning) or Thermodynamics Fireball Process on a daily basis with a prescribed metrics of substances (cow ghee, biomass, fuel, etc.) mitigates the intensities of hurricanes, tornadoes, and storms within the proximity.

4. Performing Biomass Combustion (Burning) or Thermodynamics Fireball Process on a daily basis with a prescribed metrics of substances (cow ghee, biomass, fuel, etc.) constitutes favorable conditions for clean and abundant rainfall that has less Total Dissolved Solids (TDS), and pH levels within 6 to 8, reversing drought conditions, and restricting wildfires within the proximity.

5. The ash collected from the process conducted in claims 1, 2, 3, and 4 can be mixed with waterbodies to reduce Biological Oxygen Demand (BOD) and improve the water quality, can be spread in agricultural lands to improve soil quality within the proximity.

6. Claims 1, 2, 3, and 4 use the Combustion Chamber (Brazier) with its paraphernalia as described in the Drawing Details with dimensions that depend on the purpose.
